(19) United States
(12) Patent Application Publication
(10) Pub. No.: US 2002/0072803 A1
(43) Pub. Date: Jun. 13, 2002

(54) Metatarsophalangeal resurfacing joint

(76) Inventors: Gerald Anthony Briden Saunders, Sydenham (CA); Charles Sorbie, Kingston (CA)

Correspondence Address: SPENCER, FANE, BRITT & BROWNE, 1000 WALNUT STREET, SUITE 1400, KANSAS CITY, MO, 64106-2140, US

(21) Appl. No.: 09/997,343

(22) Filed: Nov. 29, 2001

(30) Foreign Application Priority Data
Nov. 29, 2000 (CA) 2,327,199

(51) Int. Cl. A61F 2/42

(52) U.S. Cl. 623/21.19

(57) ABSTRACT

There is described an implant for resurfacing a damaged base portion of a proximal phalanx of a great toe in a metatarsophalangeal joint. The implant comprises an ovoid shaped base plate having a concave proximal bearing surface reciprocal to the shape of a head of a metatarsal and a flat rear surface, and a stem shaped anatomically to an intramedulary canal of the proximal phalanx, and the stem projects from the center of the base plate at an anatomical angle following the anatomic angular relation between a shaft and the base portion of the proximal phalanx.

### FIELD OF THE INVENTION

The invention relates to an implant that resurfaces a bone, in particular the proximal phalanx of the great toe in the metatarsophalangeal.

### BACKGROUND OF THE INVENTION

Great toe implant devices have been used to replace the defective natural metatarsophalangeal (MTP) joints. Hemi-joint replacement is generally preferred to full joint replacement when the proximal phalanx in the great toe still has good bone stock and the first metatarsal has an integral head. The implants are used to remove pain in the first metatarsal joint incurred from inflammatory arthritis and to restore joint kinetics by replacing the damaged base portion of the proximal phalanx.

U.S. Pat. No. 5,326,366 to Pascerella et. al. discloses an implant device which has an elliptical base with a concave bearing surface that has an anatomically shaped proximal articular surface with an enlarged build-up on the lateral end thereof, and an elongated stem extends distally from the seating surface of the base which includes an array of fins together having a cruciate-shaped cross-section.

U.S. Pat. No. 5,725,585 to Zobel shows a phalangeal component that has an anatomically correct concave bearing surface, a stem that is trapezoid in cross-section, and spikes on the rear surface of the implant engaging in the proximal phalanx, preventing rotation of the implant.

A proximal phalanx implant in any of the prior art has a stem projecting perpendicular from the centre of the base. This is anatomically incorrect when the implant is inserted into the bone. The proximal base of the proximal phalanx has an angular relation to the shaft of the proximal phalanx. As the result, the maximum stability of the implant can not be achieved.

In addition, many of the prior art implants have stems that are pyramidal, or are substantially three-sided, square or rectangular in cross section to prevent the rotation of the implants and provide a better stability of the implants. However, these implants require a great deal of precision and expertise for the surgeon to place the implant in the correct rotational position. Further, the implants tend to rotate after they have been implanted for a period of time.

There is therefore a need for the development of an improved great toe implant that provides maximum stability and optimal fixation to the bone for a long period of time and easier for the surgeon to insert the implant.

### SUMMARY OF THE INVENTION

It is an object of the present invention to provide an anatomically correct metatarsophalangeal implant that is an improvement of over the prior art.

According to the present invention, there is provided an implant for resurfacing a damaged base portion of a proximal phalanx of a great toe in a metatarsophalangeal joint comprising: a base plate having a concave bearing surface shaped reciprocally to a head of a metatarsal and a flat rear surface opposite the bearing surface abutting a resected base portion of the proximal phalanx; a stem projects from the centre of the base plate at an anatomical angle, the stem is shaped anatomically to an intramedulary canal of the proximal phalanx.

The present metatarsophalangeal implant enjoys a number of advantages and improvements over the prior art. For instance, the stability of the implant has been increased by anatomically shaping the stem to the intramedulary canal of the proximal phalanx. An anatomically shaped stem conforms to the endosteal cortex in the shaft, and hence maximizes the stability and optimizes the fixation of the implant to the bone. The bearing surface of the implant is angulated to align with the articulating surface of the proximal phalanx to maintain proper biomechanics with the joint. The base plate is positioned at an anatomical angle with the stem following the anatomic angular relation between the shaft and the proximal base of the proximal phalanx, which further increases the stability of the implant secured in the bone. The stem is grit blasted to further improve the adhesion of the stem to bone cements, which prevents axial rotation and inhibits pull-out of the stem itself.

Other advantages, object and features of the present invention will be readily apparent to those skilled in the art from a review of the following detailed description of the preferred embodiments in conjunction with the accompanying drawings and claims.

### BRIEF DESCRIPTION OF THE DRAWINGS

Preferred embodiments of the present invention will now be described in greater detail and will be better understood when read in conjunction with the following drawings, in which:

FIG. 1 is a perspective view of an implant in accordance with the present invention;

FIG. 2 is a side elevational view of the implant;

FIG. 3 is a top plane view of the implant;

FIG. 4 is a partial cut-away skeletal representation of a side view of the implant in accordance when surgically implanted in the proximal phalanx.

### DETAILED DESCRIPTION OF THE PREFERRED EMBODIMENTS

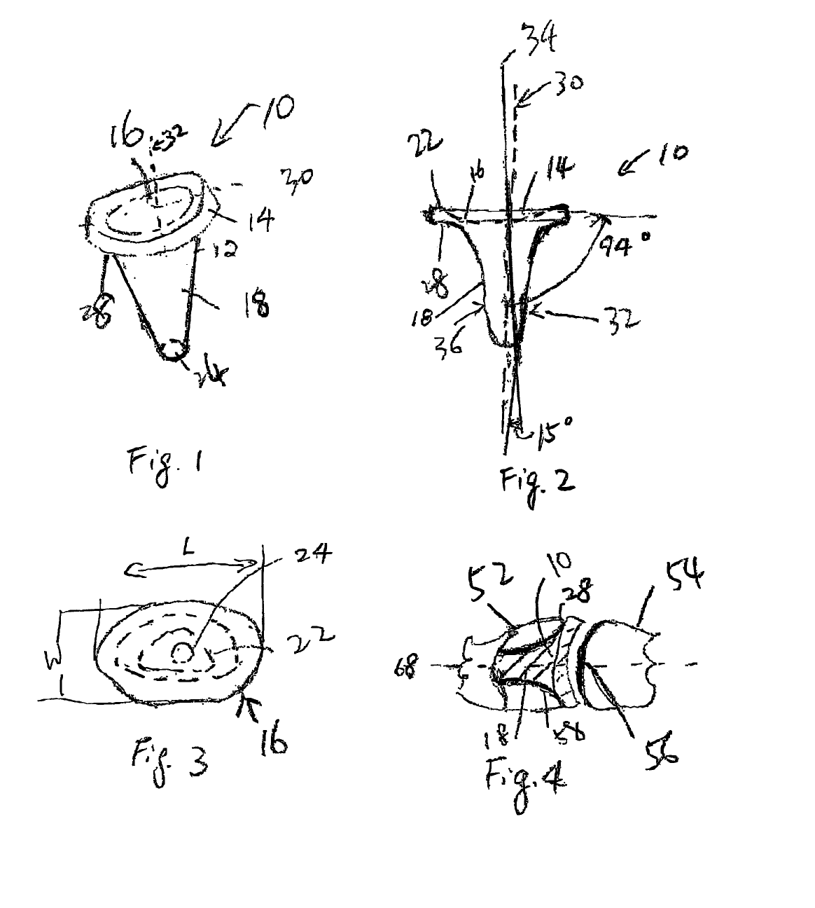

Referring to FIG. 1, a great toe implant 10 has a base plate 14 coupled to an elongated stem 18, which is adapted to be inserted in the intramedulary canal of a proximal phalanx. The base plate is ovoid shaped, having a substantially concave bearing surface 16 that is intended to contact and articulate with the head of the first metatarsal in the great toe joint, and a flat rear surface 28 opposite the concave bearing surface. A stem 18 projects from the rear surface 28 of the base plate away from the concave bearing surface 16 at an angle. The stem 18 is anatomically shaped to an intramedulary canal of a proximal phalanx. As illustrated in the top plan view of the stem in FIG. 3, the stem 18 has a general gibbosity shape 22, with the extent of the gibbosity decreasing as the stem is further away from the base plate 14 whereby the bottom 18 of the stem is almost shaped in a circle.

FIG. 2 is the side elevational view of implant 10 in accordance with the preferred embodiment, which shows a substantially symmetrical base 14 plate and a stem 18 asymmetrical about the longitudinal axis 34 extending therefrom. The stem has a side 32 inclined with respect to the longitudinal axis 34 by about 15°. The centre 30 of the stem is angularly inclined by about 4° relative to the longitudinal axis 34 (about 94° relative to the base plate), following the angular relationship of the shaft to the proximal base of the proximal phalanx. The bearing surface 16 of the base plate has a rounded edge 22 around its periphery.

The concave bearing surface 16 has a first radius of curvature along the vertical minor axis 32 and a second radius of curvature along the horizontal major axis 30. The appropriate lengths of a radii of curvature for the first and second radii respectively for different sizes of implants are as follows: Small—about 0.504 in and about 0.325 in; Medium—about 0.535 in and about 0.374 in; Large—about 0.567 in and about 0.433 in. The corresponding lengths (L) and widths (W) (see FIG. 3) of the small, medium and large base plates are 0.65 in and 0.512 in; 0.728 in and 0.556 in; and 0.827 in and 0.63 in. The length of the stem must be sufficient to allow the implant to be anchored into the proximal phalanx. The lengths of the stem of the implant for different sizes of implants are as follows: Small—about 0.485 in; Medium—about 0.563 in; Large—about 0.645 in. The thicknesses of the base plates for different sizes of implants are as follows: Small—about 0.079 in; Medium—about 0.079 in; Large—about 0.098 in. The two sides 32 and 36 of the stem projects form an angle about 22° at the top portion of the gibbosity and form an angle about 31° at the base portion of the gibbosity.

Referring to FIG. 4, a great toe joint comprises a proximal phalanx 52 that has an implant 10 surgically implanted. Typically, a cut normal to the longitudinal axis 68 of the proximal phalanx at the proximal base is made to accommodate the implant which will replace the degenerated portion of a proximal phalanx. The rear surface 28 of the stem is placed against the resected proximal base of the phalanx 52 and an anatomically shaped stem 18 is implanted into the bone canal 58 of the proximal phalanx, and conforms to the cortex in the shaft.

Metatarsal bone 54 has a rounded head portion 56. In the preferred used of the implant 10, metatarsal head is the natural surface of metatarsal bone 54. However, it is equally within the scope of the invention to use implants on metatarsal bone 54 that provided a rounded artificial surface that articulates on implant 10.

The bearing surface 16 of the base plate 14 has a complex concave surface reciprocal to the anatomically convex head 56 of the metatarsal 54. Hence, the implant 10 provides a functional advantage by increasing surface contact between the bearing surface and consequently reducing localized loading between the implant 10 and the metatarsal 54.

Preferably, the stem of the implant is grit blasted to improve adhesion to bone cement.

The implant is preferably made from biocompatibility material Cobalt-28 chromium-6 molybdenum alloy. The bearing surface of the implant is highly polished to minimize wear.

Numerous modifications, variations and adaptations may be made to the particular embodiments of the invention described above without departing from the scope of the invention, which is defined in the claims. Moreover, it will be understood that the invention described herein is not limited to implants for MTP joints. The structures as described, modified appropriately, are suitable for use in resurfacing other joints as well.

The embodiments of the invention in which an exclusive property or privilege is claimed are defined as follows:

1. An implant for resurfacing a damaged base portion of a proximal phalanx of a great toe in a metatarsophalangeal joint comprising:
a base plate having a concave bearing surface shaped reciprocally to a head of a metatarsal bone and a flat rear surface opposite the bearing surface abutting a resected base portion of said proximal phalanx;
a stem projecting from the centre of said base plate at an anatomical angle, said stem is shaped anatomically to an intramedulary canal of said proximal phalanx.

2. The implant of claim 1 wherein said base plate has an ovoid shape defining a major and a minor axis.

3. The implant of claim 1 wherein said stem is angularly inclined to said base by about 94°, following anatomic angular relation between a shaft and said base portion of said proximal phalanx.

4. The implant of claim 3 wherein said stem conforms to the cortex in a shaft of said proximal phalanx.

5. The implant of claim 4 wherein said stems is grit blasted to allow the implant to adhere to bone cements.

6. The implant of claim 1 wherein said implant is made of Co—Cr biocompatible material.
